(19) United States
(12) Patent Application Publication
(10) Pub. No.: US 2026/0120847 A1
(43) Pub. Date: Apr. 30, 2026

(54) CAUSAL EFFECT ESTIMATION METHOD FOR TREATMENT DECISION SUPPORT OF INTRACRANIAL HEMORRHAGE USING IMAGING AND NON-IMAGING DATA

(71) Applicant: The Chinese University of Hong Kong, Hong Kong (CN)

(72) Inventors: Qi DOU, Hong Kong (CN); Wenao MA, Hong Kong (CN); Jill ABRIGO, Hong Kong (CN); Calvin Hoi-Kwan MAK, Hong Kong (CN)

(21) Appl. No.: 19/004,745

(22) Filed: Dec. 30, 2024

Related U.S. Application Data
(60) Provisional application No. 63/616,209, filed on Dec. 29, 2023.
(60) Provisional application No. 63/562,525, filed on Mar. 7, 2024.

(51) Int. Cl. G16H 30/40; G06T 7/00; G06V 10/77

(52) U.S. Cl. CPC G16H 30/40; G06T 7/0012; G06V 10/7715; G06T 2207/20081; G06T 2207/30004

(57) ABSTRACT

The subject invention pertains to a novel causal effect estimation approach for aneurysmal subarachnoid hemorrhage treatment and treatment related decision support based on multi-modality imaging and non-imaging clinical data. The provided embodiments are shown to effectively estimate the treatment effect; and to utilize multi-modality data to perform this estimation. Embodiments provide a novel scheme that uses multi-modality confounders distillation architecture to predict the treatment outcome and treatment assignment simultaneously. The distilled confounder features provide an imaging and non-imaging interaction representation learning strategy to use the complementary information extracted from different modalities to balance the representation distribution of different treatment groups. Extensive experiments have been performed on a subarachnoid hemorrhage clinical dataset, which is collected from the Hospital Authority Data Collaboration Lab and contains 656 cases. The provided method shows consistent improvements on the evaluation metrics of treatment effect estimation, achieving state-of-the-art results over related art competitors.

### CROSS-REFERENCE TO RELATED APPLICATION

The present application claims the benefit of U.S. Provisional Application Ser. No. 63/562,525, filed Mar. 7, 2024; and 63/616,209, filed Dec. 29, 2023; the disclosures of which are incorporated herein by reference in their entirety.

### BACKGROUND OF THE INVENTION

Aneurysmal subarachnoid hemorrhage (aSAH) is a life-threatening hemorrhagic stroke with an acute bleeding inside the brain. It is considered as the most devastating type of stroke, with early case death rate of over 30% and disability rate of about 20% among survivors [1], [2]. Interventions such as endovascular treatment and clipping, are major clinical treatments for aSAH, but the outcomes of interventions are hard to estimate and closely correlated with the pre-morbid condition and co-existing illness of each patient [2]. For example, serious results of rebleeding, caused by aneurysm recurrence, may occur after endovascular treatment for patients with hematomas [3]. The treatment decision-making for aSAH is still difficult in current clinical practice [4], [5], because the role of interventions remains unclear [6]. aSAH is a serious medical emergency of the brain that has high mortality and poor prognosis. Treatment effect estimation is of high clinical significance to support the treatment decision making for aneurysmal subarachnoid hemorrhage. Therefore, a reliable decision-making support model that can estimate the potential outcome of each type of treatment based on patients' individual conditions is highly desirable and can be significantly beneficial for optimization of aSAH management.

Most existing works on treatment outcome predictions can only predict the outcome of a certain type of treatment, but are unable to simultaneously compare the potential outcomes of different treatments for a patient [7]-[11]. Treatment outcome prediction can be transferred to a typical causal effect estimation problem [12], [13], aiming to predict the expected causal effect of applying different treatments [14]-[16]. The illustration of causal effect estimation according to an exemplary and non-limiting embodiment of the subject invention can be seen in FIG. 1. One rigorous method of making these causal effect estimations is randomized controlled trials [17]. However, well designed randomized controlled trials with special considerations to the aspect are expensive and can be difficult to conduct for the treatments in aSAH [18]. The outstanding difficulty in estimating causal effect from observational data is missing counterfactual outcomes [13], [19]-[25]. This means that one can only observe the outcome of factual individual treatment decision, thus the counterfactual information, i.e., the outcomes for treatment decisions that are not assigned to the patient, are always missing. A natural way to address this problem is to estimate the individual counterfactual outcomes based on the observed data. However, in practice, some treatments could be more likely applied to a particular group with certain covariate values [25]-[27]. For example, the endovascular treatment is more likely to be suggested to aSAH patients without evident mass effect than those with poor clinical conditions and large intraparenchymal hematomas [2], [4]. This bias in treatment assignment can make counterfactuals estimation become unreliable [13].

To achieve causal effect estimation without counterfactual information, some methods proposed to utilize matching and re-weighting scheme to make the distributions of different treatments more comparable [28]-[30]. Other methods learned to balance the representation of different treatment groups [13], [19], [22], which is one of the most effective techniques in counterfactual outcome estimation for treatment effect predictions. Although prior works have made promising progress on causal effect estimation based on single modality data, how to achieve more effective balanced representation learning from multi-modality data (e.g., imaging and non-imaging clinical data) has not been sufficiently explored. In current clinical practice, the management of aSAH highly relies on the complementary information of data collected from multiple sources, including imaging data and non-imaging clinical data, to perform diagnosis and preoperative planning. However, there is no existing effort in performing treatment outcome prediction from the perspective of causal effect estimation for aSAH management.

Alternative embodiments provide treatment outcome prediction for stroke. As accurate outcome prediction is highly desirable to optimize treatment decision-making for stroke, related art machine learning-based methods have been proposed to estimate the treatment outcome in clinical practice [31]. For instance, several variables are extracted from imaging data and non-imaging clinical data to measure the endovascular treatment outcome for acute ischemic stroke using gradient boosting classifiers [32]. Another related artwork used a deep convolutional neural network to accurately predict final lesion volume for patients treated for acute ischemic stroke [33]. Moreover, incorporating initial clinical presentations, laboratory data, and imaging findings is proved to be effective for outcome prediction in patients with primary intracerebral hemorrhage [34], and a prediction model is learned from 64 extracted variables to perform postsurgical functional outcome prediction for hypertensive intracerebral hemorrhage [6]. A typical study [8] predicted the functional outcome of aSAH treatment by using extracted clinical variables. Another work focused on developing a new scoring model using multivariate logistic regression analysis to predict functional outcome in poor-grade aSAH [10].

However, on the one hand, these related art studies can only estimate outcomes without considering the counterfactual estimation. Therefore, the previous methods are difficult to provide valuable and explainable support for decision making on treatment strategy. There is no existing solution in aSAH to make the causal effect estimation regarding the discrepancy between the different treatment strategies. On the other hand, most of the existing treatment outcome prediction models only used the tabular data to be the input, where the multi-modality information implied on imaging data are extracted manually. However, directly combining the non-imaging clinical data and imaging information into the tabular data is inconsistent with clinical practice, as clinical data have a significant impact on image interpretation [35].

### BRIEF SUMMARY OF THE INVENTION

Embodiments of the subject invention provide systems, methods, and models that can simultaneously analyze imaging and non-imaging clinical data in a manner that is advantageously beneficial for treatment outcome prediction in real-world practice.

The advantageous causal effect estimation systems and methods for treatment and/or decision support of aSAH are based on multi-modality observational data. To achieve effective causal effect estimation, embodiments incorporate an auxiliary task of treatment assignment prediction to distill the multi-modality covariates that affect both the treatment assignment and outcome, i.e., the confounders. In certain embodiments this confounder distillation extracts certain useful information and drives the model to focus on the discrepancy between the outcomes of different treatment assignments. With the distilled features, embodiments further develop an imaging and non-imaging feature interaction strategy to address the problem of missing counterfactual outcomes, which identifies similar pairs with the complementary information extracted from different modalities. The similar pairs are then used to balance the distributions of different treatment groups to achieve reliable counterfactual estimation. A clinical multi-modality dataset of subarachnoid hemorrhage is collected to validate the provided method, which contains 656 cases with both head CT images and a set of non-imaging clinical data from clinical reports. This novel approach leverages multimodality data to conduct causal effect estimation for treatment outcome predictions to support treatment and treatment related decision-making for aSAH.

Embodiments of the subject invention combine causal effect estimation and multi-modality learning to utilize the multimodality information to achieve effective outcome estimation for aSAH treatment decision support.

Embodiments provide a novel multi-modality confounders distillation for causal effect estimation from imaging and non-imaging clinical data. The provided systems and methods incorporate treatment assignment prediction as the auxiliary task to extract multi-modality covariates that affect both treatment assignment and outcome.

Embodiments provide an imaging and non-imaging interaction representation learning module, which can help to balance representation distribution with the help of complementary information extracted from different modalities.

Embodiments have been tested on a large clinical subarachnoid hemorrhage dataset collected from the Hospital Authority Data Collaboration Lab, which is of clinical significance and contains both CT data and non-imaging clinical variables. The provided method significantly outperforms existing causal effect estimation approaches in the task of treatment outcome prediction.

Embodiments advantageously focus on causal effect estimation for each individual based on observational data, which are non-randomized controlled trials thus are often readily available. Observational data of aSAH include the decision of past interventions and outcomes of each patient, along with imaging (e.g., CT scans) and non-imaging clinical data that are critical for treatment decision-making.

### BRIEF DESCRIPTION OF THE DRAWINGS

FIG. 1 shows an illustration of causal effect estimation according to an embodiment of the subject invention. Given individualized imaging data and non-imaging clinical data, embodiments estimate the outcome ŷ₀, ŷ₁, and ŷ₂, which denote the survival probability of performing conservative treatment (t₀), endovascular treatment (t₁), and clipping (t₂) respectively.

FIGS. 2A-2F show respective schematic overviews of a provided model according to different respective embodiments of the subject invention. The respective models take multimodality imaging and non-imaging data as input and output the outcome predictions for different treatment assignments. Embodiments provide multi-modality confounders distillation (e.g., grey boxes, also known as a treatment prediction module) to disentangle confounding covariates from non-confounding covariates and use imaging and non-imaging interaction representation learning (e.g., blue box) to balance distributions of different treatment groups to deal with missing counterfactual outcomes. Both confounders distillation and imaging and non-imaging interaction representation learning are modules that introduce additional information to help train better models during the training phase. Among them, the purpose of confounders distillation is to help the model extract features related to confounders more effectively. These features will also be fed into INII during the training phase, thereby outputting additional loss to better train the model, according to an embodiment of the subject invention.

FIG. 3 shows an illustration of the imaging and non-imaging interaction representation learning according to an embodiment of the subject invention. On the top box of this figure, embodiments first select nine samples a-i using the similarity information in the feature space of non-imaging data representation Ψ^{cli}. The information of the sample selection and the similarities are then transmitted to guide the distribution balancing of imaging data representation Ψ^{img}, which is shown on the bottom of this figure.

FIG. 4 illustrates the graphical model of our task in idealized scenario.

FIG. 5A is a graph showing risk and error analysis of multi-modality confounders distillation (MMCD) with regards to ∈_{ATE} and R_{POL} according to an embodiment of the subject invention.

FIG. 5B is a pair of graphs showing model reliance analysis of multi-modality confounders distillation (MMCD) with regards to feature importance according to an embodiment of the subject invention.

FIG. 6A illustrates ablation results with and without the imaging and non-imaging interaction (INII) representation learning according to certain embodiments of the subject invention.

FIG. 6B illustrates t-SNE visualizations of the learned feature representations with and without INII according to certain embodiments of the subject invention. The green, red, and blue markers denote the samples treated with endovascular treatment, clipping, and conservative treatments respectively. The arrow and black circle highlight the non-overlap regions.

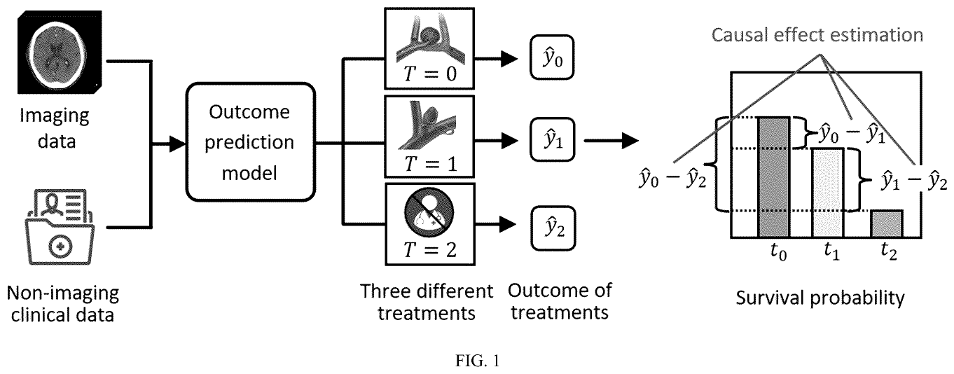

TABLE I
Comparison results with related art methods on subarachnoid hemorrhage treatment
outcome estimation on the in-house dataset; number: mean ± std (across trials).

| | Evaluation metrics | | | | | | |
|---|---|---|---|---|---|---|---|
| Method | R_{POL} ↓ | ϵ_{ATE}_{_{0.1}} ↓ | ϵ_{ATE}_{_{0.2}} ↓ | ϵ_{ATE}_{_{0.3}} ↓ | A_{CC}_{₀} ↑ | A_{CC}_{₁} ↑ | A_{CC}_{₂} ↑ |
| BNN [13] | .093 ± .067 | .194 ± .098 | .208 ± .166 | .183 ± .143 | .772 ± .055 | .892 ± .083 | .893 ± .097 |
| CFR-MMD [19] | .085 ± .109 | .137 ± .101 | .174 ± .155 | .133 ± .134 | .775 ± .078 | .910 ± .075 | .872 ± .119 |
| CFR-WASS [19] | .081 ± .059 | .123 ± .084 | .187 ± .127 | .145 ± .152 | .782 ± .075 | .891 ± .083 | .882 ± .107 |
| SITE [22] | .073 ± .076 | .132 ± .088 | .185 ± .147 | .135 ± .117 | .789 ± .086 | .908 ± .058 | .909 ± .106 |
| Dragonnet [24] | .078 ± .065 | .149 ± .085 | .161 ± .145 | .159 ± .153 | .781 ± .088 | .910 ± .072 | .884 ± .111 |
| β-Intact-VAE [25] | .069 ± .061 | .133 ± .094 | .163 ± .143 | .118 ± .124 | .780 ± .102 | .908 ± .067 | .901 ± .115 |
| DAFT [46] | .094 ± .067 | .136 ± .096 | .219 ± .150 | .184 ± .129 | .780 ± .104 | .926 ± .069 | .917 ± .123 |
| FiLM [47] | .096 ± .080 | .184 ± .102 | .242 ± .152 | .195 ± .149 | .787 ± .106 | .919 ± .066 | .920 ± .092 |
| Interactive-Model [48] | .108 ± .087 | .177 ± .131 | .294 ± .110 | .173 ± .103 | .784 ± .098 | .913 ± .076 | .911 ± .104 |
| Embodiment | .058 ± .053 | .112 ± .085 | .141 ± .132 | .114 ± .124 | .793 ± .082 | .924 ± .071 | .917 ± .094 |

TABLE II
Comparison results with other model structures on subarachnoid
hemorrhage treatment outcome estimation on the in-house
dataset; number: mean ± std (across trials).

| | Evaluation metrics | | |
|---|---|---|---|
| Method | R_{POL} ↓ | ϵ̅_{ATE} ↓ | Ācc ↑ |
| W/o separate heads | .067 ± .056 | .124 ± .072 | .859 ± .049 |
| Embodiment | .058 ± .053 | .122 ± .067 | .878 ± .048 |

TABLE III
Treatment outcome prediction with different data modalities.
The ϵ̅_{ATE} is the average of ϵ_{ATE0, 1}, ϵ_{ATE0, 2}, and
ϵ_{ATE1, 2}. The Ācc is the average of Acc₀, Acc₁, and Acc₂.

| Modality | | Evaluation metrics | | |
|---|---|---|---|---|
| Image | Clinical | R_{POL} ↓ | ϵ̅_{ATE} ↓ | Ācc ↑ |
| ✓ | | .106 ± .088 | .222 ± .070 | .836 ± .053 |
| | ✓ | .098 ± .091 | .176 ± .078 | .844 ± .062 |
| ✓ | ✓ | .058 ± .053 | .122 ± .067 | .878 ± .048 |

TABLE IV
Ablation results for the two components, i.e., MMCD and INII
in the provided method. The ϵ̅_{ATE} is the average of ϵ_{ATE0, 1},
ϵ_{ATE0, 2}, and ϵ_{ATE1, 2}. The Ācc is the average of
Acc₀, Acc₁, and Acc₂.

| | |
|---|---|
| Method | Evaluation metrics |

| MMCD | BRL | R_{POL} ↓ | ϵ̅_{ATE} ↓ | Ācc ↑ |
|---|---|---|---|---|
| | | .101 ± .037 | .174 ± .082 | .839 ± .056 |
| ✓ | | .084 ± .048 | .163 ± .081 | .851 ± .056 |
| | ✓ | .074 ± .042 | .152 ± .084 | .865 ± .055 |
| ✓ | ✓ | .058 ± .053 | .122 ± .067 | .878 ± .048 |

### DETAILED DISCLOSURE OF THE INVENTION

The embodiments of subject invention show a machine learning system and methods for providing treatment decision support from imaging data and clinical data with causal effect estimation and a known number of treatment options. The terminology used herein is for the purpose of describing particular embodiments only and is not intended to be limiting of the invention. As used herein, the term “and/or” includes any and all combinations of one or more of the associated listed items. As used herein, the singular forms “a,” “am,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention pertains. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

When the term “about” is used herein, in conjunction with a numerical value, it is understood that the value can be in a range of 90% of the value to 110% of the value, i.e. the value can be +/−10% of the stated value. For example, “about 1 kg” means from 0.90 kg to 1.1 kg.

In describing the invention, it will be understood that a number of techniques and steps are disclosed. Each of these has individual benefits and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

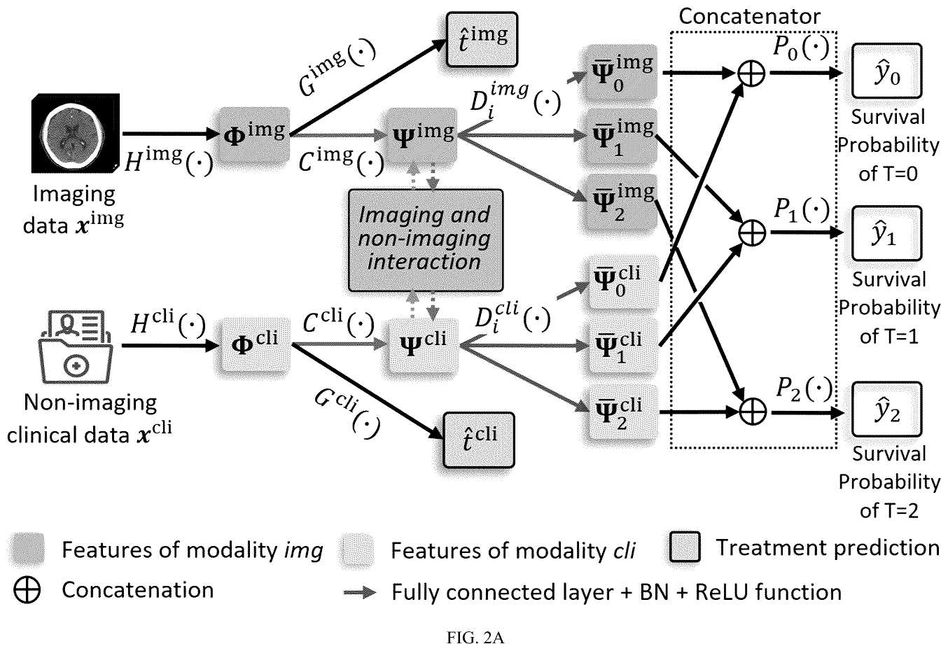

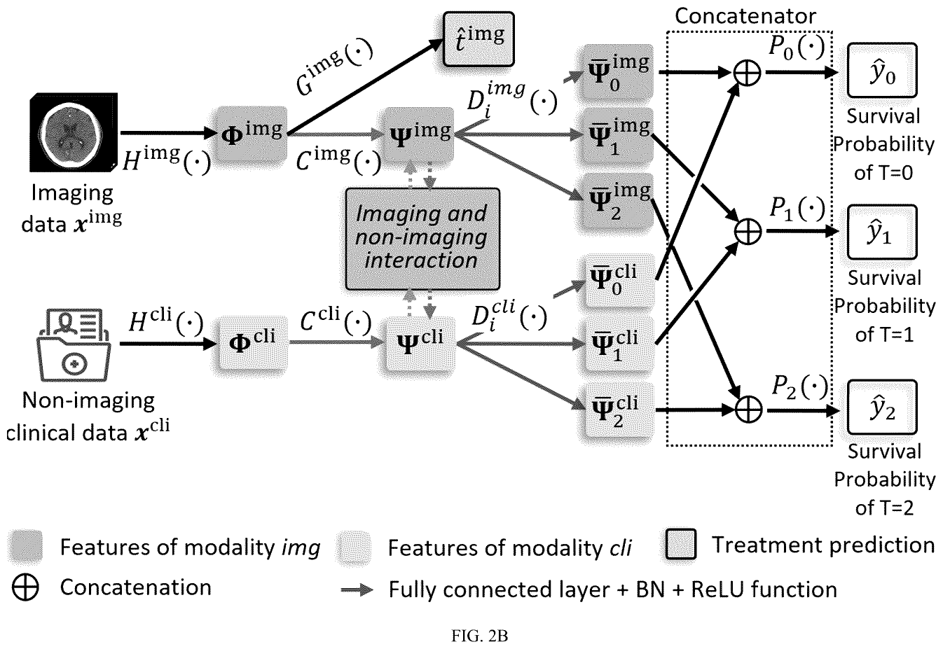

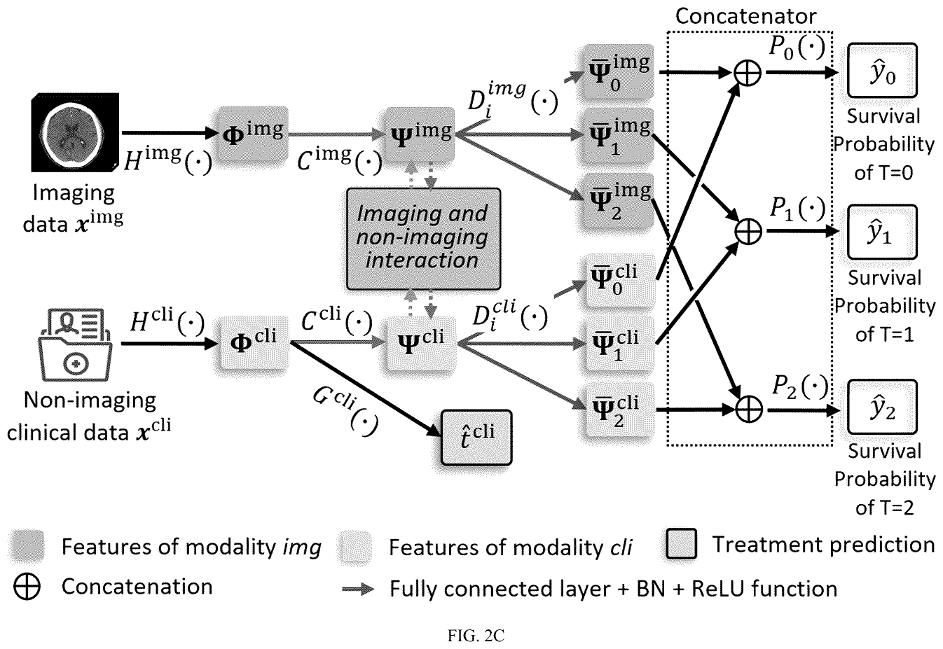

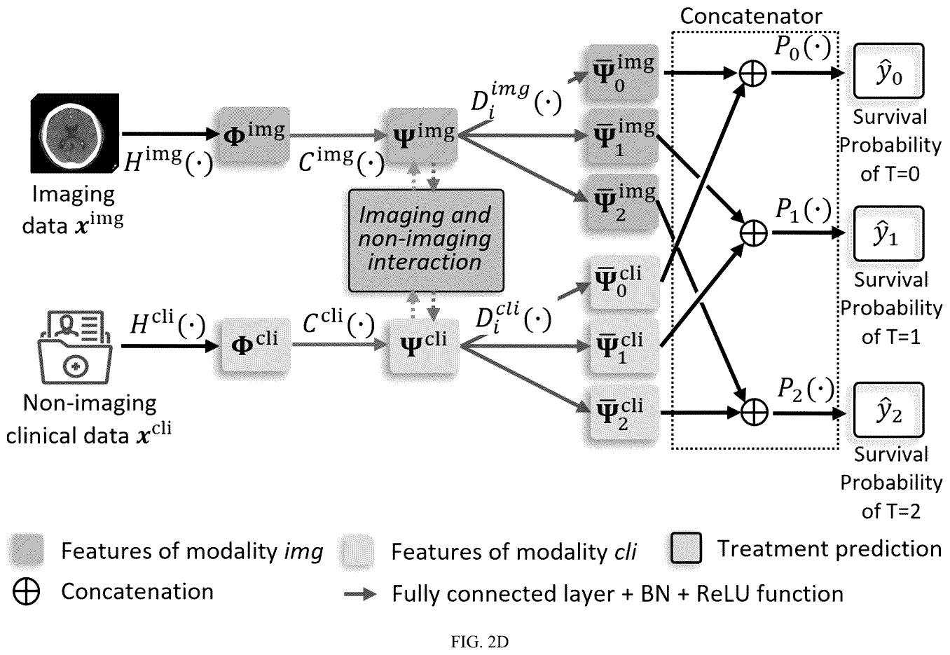

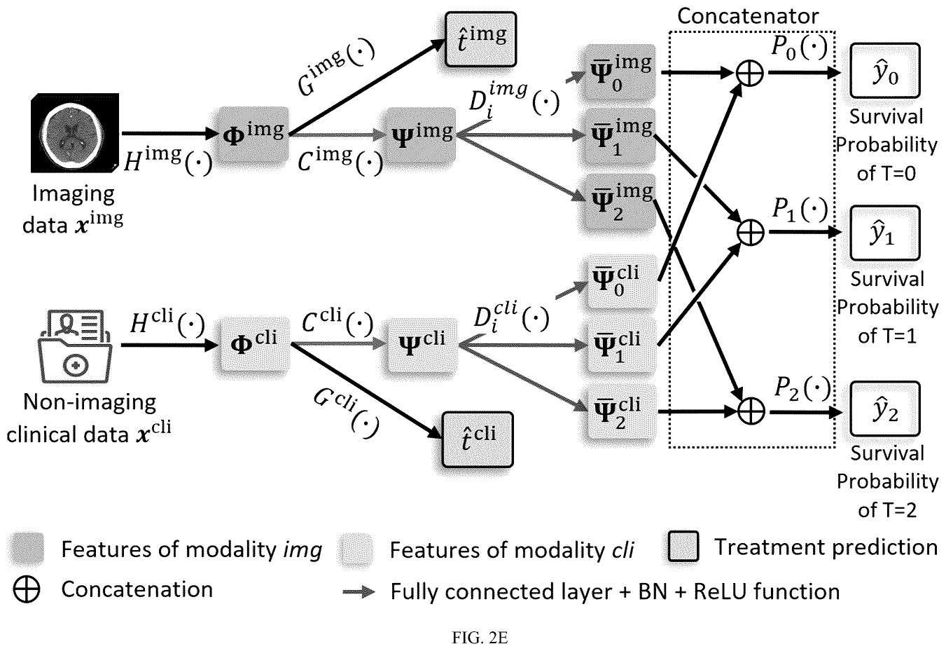

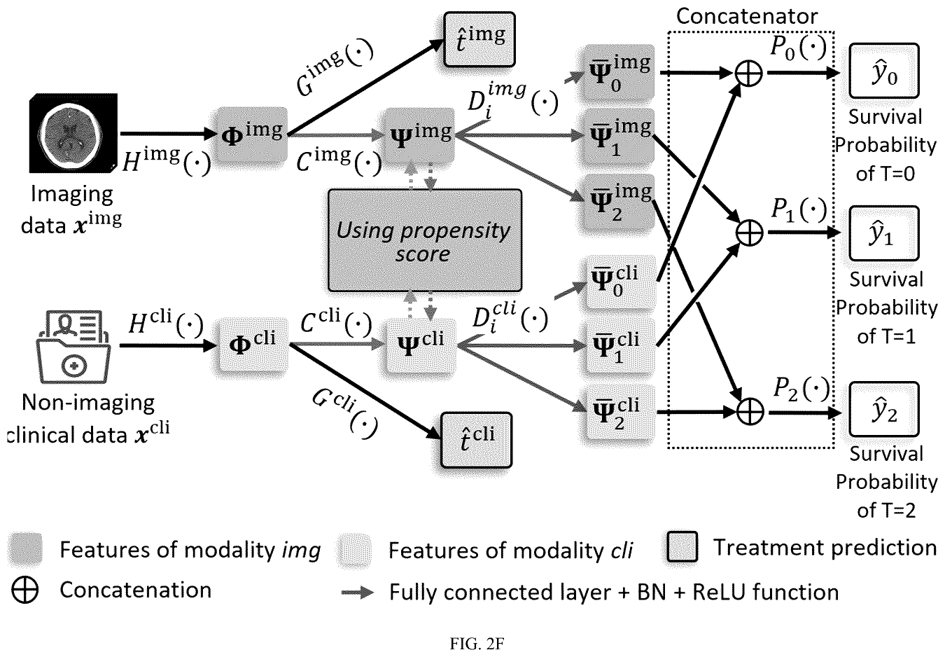

Certain embodiments start with the formulation of treatment effect estimation, then describe the provided multimodality confounders distillation component and the imaging and non-imaging interaction representation learning component, and finally give a model explanation with causal graph analysis on the provided method. FIGS. 2A-2F present an overview of one such embodiment of a provided method.

Embodiments estimate treatment effect given a set of observations consisting of the factual treatment T, observed covariates X, and factual outcome Y. Let t∈{0, 1, 2} be the potential treatments that can be assigned to the patients, e.g., the conservative treatment, endovascular treatment or clipping in this study, and Y (t) be the potential treatment outcomes, e.g., whether the patient would survive or die in one year with treatment t. Let x=(x^{img}, x^{cli}) be the observed covariates of a patient where x^{img} and x^{cli} denote the imaging and non-imaging clinical information respectively. One goal is to conduct individualized causal effect estimation, e.g., to estimate the treatment effect conditioned on x:

$${τ}_{0 , 1} ( x ) = 𝔼 [ Y ( 0 ) \vert X = x ] - 𝔼 [ Y ( 1 ) \vert X = x ] , \qquad ( 1 )$$

$${τ}_{0 , 2} ( x ) = 𝔼 [ Y ( 0 ) \vert X = x ] - 𝔼 [ Y ( 2 ) \vert X = x ] , \qquad ( 2 )$$

$${τ}_{1 , 2} ( x ) = 𝔼 [ Y ( 1 ) \vert X = x ] - 𝔼 [ Y ( 2 ) \vert X = x ] . \qquad ( 3 )$$

One point of certain embodiments is that, in a set of observations which is used to train the model, embodiments can never observe the counterfactual outcomes that would have been if the clinician had made an alternative decision. For instance, if T=0, then the potential treatment outcomes Y (1) and Y (2) are counterfactual outcomes that are unobservable.

To assure the individualized causal effect can be successfully estimated, the following assumptions are widely adopted by related art counterfactual estimation methods [12]: 1) The potential outcomes Y (0), Y (1), and Y (2) of one unit do not vary with the treatment of any other units. Moreover, each treatment does not have multiple forms or versions that can lead to different potential outcomes (Stable Unit Treatment Value Assumption); 2) the factual treatment assignment T is independent of potential outcome Y (t), given the covariates X: T [symbol] Y (t)|X (Ignorability); 3) for any unit with covariates x, any treatment t∈{0, 1, 2} is possible to be assigned on the data: P(T=t|X=x)>0 (Positivity).

Embodiments provide casual effect estimation with multi-modality confounders distillation. As can be seen in FIG. 2A, this embodiment of the provided causal effect estimation framework uses two parallel networks to receive the imaging data x^{img} and non-imaging clinical data x^{cli} respectively. The input data are forwarded to two feature extraction networks H^{img}(⋅) and H^{cli}(⋅), generating two representations Φ^{img} and Φ^{cli}. Then, embodiments use two separate network branches for each modality. One branch aims to conduct treatment assignment prediction (discussed below), and the other branch aims to estimate the treatment outcome. In order to conduct causal effect estimation, a fully connected layer with a batch normalization and activation function ReLU have been used to generate the representations Ψ^{img} and Ψ^{cli} after the feature extraction network. Then, separate heads are used to estimate the outcome of T=0, T=1 and T=2. Specifically, the representations

$${\overline{Ψ}}_{0}^{img} , {\overline{Ψ}}_{1}^{img} , {\overline{Ψ}}_{2}^{img} , {\overline{Ψ}}_{0}^{cli} , {\overline{Ψ}}_{1}^{cli} , and {\overline{Ψ}}_{2}^{cli}$$

are generated from Ψ^{img} and Ψ^{cli} by severally using a fully connected layer followed by a batch normalization layer and ReLU function. The representations

$${\overline{Ψ}}_{0}^{img} and {\overline{Ψ}}_{0}^{cli}$$

are concatenated to generate the final outcome of treatment

$$T = 0 : {X}^{{\hat{y}}_{0}} = {P}_{0} ( {\overline{Ψ}}_{0}^{img} , {\overline{Ψ}}_{0}^{cli} ) ,$$

using a fully connected layer followed by a Sigmoid function. The final outcome of

$${\hat{y}}_{1} = {P}_{1} ( {\overline{Ψ}}_{1}^{img} , {\overline{Ψ}}_{1}^{cli} ) and = {\hat{y}}_{2} = {P}_{2} ( {\overline{Ψ}}_{2}^{img} , {\overline{Ψ}}_{2}^{cli} )$$

are obtained in the same way. The loss of treatment outcome prediction [symbol] ₒₚ can be calculated as:

$${ℒ}_{op} = \frac{1}{N} [ {\sum }_{n ∈ {S}_{0}} BCE ( {\hat{y}}_{0 , n} , {y}_{0 , n} ) + \\ {\sum }_{n ∈ {S}_{1}} BCE ( {\hat{y}}_{1 , n} , {y}_{1 , n} ) + {\sum }_{n ∈ {S}_{2}} BCE ( {\hat{y}}_{2 , n} , {y}_{2 , n} ) ] , \\ \qquad ( 4 )$$

where the subscript n denotes the n-th sample, BCE denotes the binary cross entropy function, S₀, S₁, and S₂ denote the set of samples treated with the treatment 0, 1, and 2 respectively in the mini-batch samples, and N denotes the number of samples in each mini-batch.

For the task of causal effect estimation, the features extracted from the observational data x that affect both treatment assignment and treatment outcome are crucially important [37]. These features are so called confounders. If the model fails to adjust for confounders, the outcome estimation can highly depend on the parts of x that are irrelevant for the treatment assignment, which hurts the performance of causal effect estimation [24]. Moreover, the high dimension of imaging data and the nonstandard distribution of covariates raise the bar of distilling the confounders from the imaging data. To this end, embodiments provide a multi-modality confounders distillation component, which performs the treatment assignment prediction, i.e., predicting the treatment assignment, and treatment outcome prediction simultaneously. With regards to the treatment assignment prediction, one attempts to disentangle the groups of different treatment strategies. On the contrary, with regard to causal effect estimation, one attempts to balance the treated and control distributions. The two objectives may be in conflict. Hence, embodiments use the features of the earlier layer to perform the treatment assignment prediction, and then provide an additional module for final treatment outcome prediction. Specifically, embodiments forward the representations Φ^{img} and Φ^{cli} to two prediction networks G^{img}(⋅) and G^{cli}(⋅), which both contain a fully connected layer and a Sigmoid function, yielding the predicted treatment assignments \hat{t}^{img} and \hat{t}^{cli} respectively. The loss of treatment assignment prediction [symbol] ᵤₚ can be calculated as:

$${ℒ}_{ap} = \frac{1}{N} {\sum }_{n ∈ N} [ BCE ( {\hat{t}}_{n}^{img} , {t}_{n} ) + BCE ( {\hat{t}}_{n}^{cli} , {t}_{n} ) ] , \qquad ( 5 )$$

where BCE denotes the binary cross entropy function, tₙ denotes the factual treatment assignment and subscript n denotes the n-th sample.

As the provided model in certain embodiments is estimating the individual treatment outcome instead of treatment assignment prediction, embodiments consider the treatment assignment prediction as an auxiliary task, which can further improve the model performance. With the help of the treatment prediction module (e.g., as shown in FIGS. 2A-2F), the features extraction network H^{img} and H^{cli} can highlight the information of the confounders, which are the parts of inputs affecting both the treatment assignment and treatment outcome. Thereby, the confounders of each modality can be distilled from the original inputs, while the factors which are irrelevant for the treatment can be attenuated.

Embodiments provide imaging and non-imaging interaction for balanced representation learning. One main difficulty of causal outcome estimation is that embodiments can never observe the counterfactual outcome on the training set, while the training set is usually biased as some treatments could be more likely applied to some particular groups in clinical practice. For instance, if embodiments only observe the outcomes of aSAH patients who have large hematoma treated with clipping, this can inhibit the training of a model to estimate outcomes of patients with large hematoma treated with other treatments (e.g., conservative treatment and endovascular treatment). To tackle this problem, certain provided systems and methods advantageously apply the strategy of using balanced representation learning to increase the overlap between distributions of different treatment groups [19], [22], [24], [25]. If the factual treatment of a training sample T is 0 so that only Y (0) is observed, embodiments can refer to another sample which has different treatment strategy but is “similar” in general condition to that sample (e.g., the sample whose factual treatment T is 1 or 2). If the covariates of these two samples are highly similar, their positions in the representation spaces of Ψ^{img} and Ψ^{cli} should be closer to each other, so that embodiments can achieve reliable counterfactual outcome estimation.

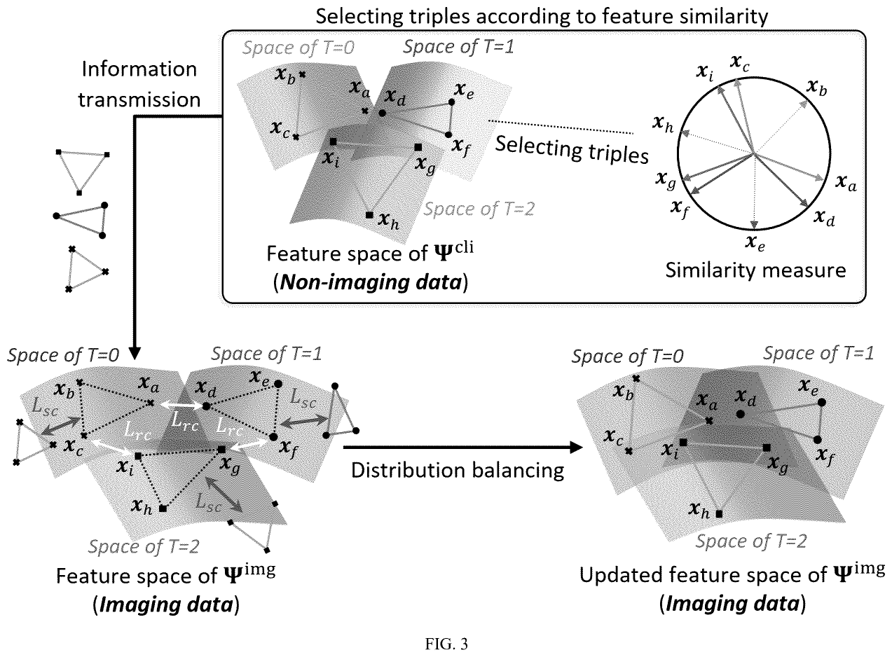

One challenge is how to measure similarity and how to find the “similar” pairs. To achieve that, embodiments provide an imaging and non-imaging feature interaction strategy that uses the representations from another modality to measure the similarity. As can be seen in FIG. 3, embodiments provide this imaging and non-imaging interaction (INII) for balanced representation learning in representation of imaging data Ψ^{img} for instance. Recall that S₀, S₁, and S₂ denote the set of training samples treated with the conservative treatment, endovascular treatment, and clipping respectively in the mini-batch. To begin with, embodiments find the target pairs by selecting the samples which have different treatment assignments but are closest to each other in the feature space of the other modality, e.g., Ψ^{cli} in this example:

$$( a , d ) = {arg max}_{a ∈ {S}_{o} , d ∈ {S}_{1}} {Ψ}_{a}^{cli} \cdot {Ψ}_{d}^{cli} / ‖ {Ψ}_{a}^{cli} ‖ \times ‖ {Ψ}_{d}^{cli} ‖ , \qquad ( 6 )$$

$$( f , g ) = {arg max}_{f ∈ {S}_{1} , g ∈ {S}_{2}} {Ψ}_{f}^{cli} \cdot {Ψ}_{g}^{cli} / ‖ {Ψ}_{f}^{cli} ‖ \times ‖ {Ψ}_{g}^{cli} ‖ , \qquad ( 7 )$$

$$( c , i ) = {arg max}_{c ∈ {S}_{o} , i ∈ {S}_{2}} {Ψ}_{c}^{cli} \cdot {Ψ}_{i}^{cli} / ‖ {Ψ}_{c}^{cli} ‖ \times ‖ {Ψ}_{i}^{cli} ‖ , \qquad ( 8 )$$

where a, c, d, f, g, and i denote indexes of selected samples. Then, embodiments randomly select one data point from each treatment group: x_{b}, xₑ, and xₕ. Notably, embodiments can introduce some stochastic factors in this place to avoid selecting the same pair repeatedly during the training phase. At last, three triplets can be obtained from the conservative treatment group, endovascular treatment group, and clipping group (xₐ, x_{b}, x_{c}), (x_{d}, xₑ, x_{f}), and (x_{g}, xₕ, xᵢ). One goal is to close the distance of similar sample with different treatment, e.g., the pairs (xₐ, x_{d}), (x_{f}, x_{g}), and (x_{c}, xᵢ). Therefore, embodiments construct an additional representation closing loss:

$${ℒ}_{rc}^{img} = \frac{1}{3} ( {‖ Proj ( {Ψ}_{a}^{img} ) - Proj ( {Ψ}_{d}^{img} ) ‖}_{2}^{2} + {‖ Proj ( {Ψ}_{f}^{img} ) - Proj ( {Ψ}_{g}^{img} ) ‖}_{2}^{2} + {‖ Proj ( {Ψ}_{c}^{img} ) - Proj ( {Ψ}_{i}^{img} ) ‖}_{2}^{2} ) , \qquad ( 9 )$$

where Proj(⋅) denotes a projection head network in [43]. Moreover, embodiments further preserve the local similarity between the samples with the same treatment (e.g., xₐ, x_{b}, and x_{c}) in the space of img. Specifically, embodiments measure the similarity via Cosine similarity and calculate the distance between the similarities of Ψ^{img} and Ψ^{cli}. For instance, the squared distance between the similarities of xₐ and x_{c} can be expressed by:

$${{D}_{i m g}^{2} ( a , c ) = ( {S}_{C} ( Proj ( {Ψ}_{a}^{cli} ) , Proj ( {Ψ}_{c}^{cli} ) ) - {S}_{C} Proj ( {Ψ}_{a}^{i m g} ) , Proj ( {Ψ}_{c}^{i m g} ) ) )}^{2} , \qquad ( 10 )$$

where S_{C} denotes cosine similarity. The cosine similarity can be expressed by:

$${S}_{C} ( {v}_{1} , {v}_{2} ) = \frac{{v}_{1} \cdot {v}_{2}}{{‖ {v}_{1} ‖}_{2} \times {‖ {v}_{2} ‖}_{2}} , \qquad ( 11 )$$

where v₁ and v₂ are two vectors. Then, embodiments maintain that the similarity between these sample are consistent by adding a similarity consistency loss:

$${L}_{s c}^{i m g} = \frac{1}{9} [ {D}_{img}^{2} ( a , c ) + {D}_{i m g}^{2} ( a , b ) + {D}_{img}^{2} ( b , c ) + {D}_{img}^{2} ( d , e ) + {D}_{img}^{2} ( d , f ) + {D}_{i m g}^{2} ( e , f ) + {D}_{img}^{2} ( g , h ) + {D}_{img}^{2} ( g , i ) + {D}_{i m g}^{2} ( h , i ) ] . \qquad ( 12 )$$

As the similarity information are obtained from another modality, this process can be seen as a way of information transmission. Similarly, the representation closing loss and similarity consistency loss of clinical non-imaging modality

$$( {ℒ}_{rc}^{cli} and {ℒ}_{sc}^{cli} )$$

can be calculated by the same way mentioned above.

Embodiments advantageously improve the model by summarizing all the losses mentioned above:

$$ℒ = {ℒ}_{op} + {ℒ}_{ap} + {ℒ}_{rc}^{img} + {ℒ}_{rc}^{cli} + {ℒ}_{sc}^{img} + {ℒ}_{sc}^{cli} + λ {‖ W ‖}_{2} , \qquad ( 13 )$$

where λ denotes the weight of the L₂ regularization of model parameters W, which is set as 10⁻⁵ in the provided implementation.

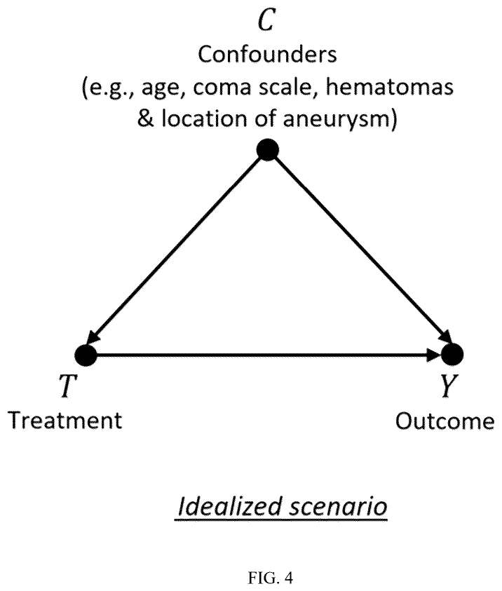

Embodiments provide a causal graph of the provided model. Certain embodiments use causal graphs to show the insight of the provided model. As can be seen in FIG. 4, in an idealized scenario, embodiments can observe all the confounders C that affect both treatment T and outcome Y. This makes it easy to estimate the causal effect [20]. However, in realistic scenarios, the patient's health status U cannot be directly measured, or can even undefined. Fortunately, such unobserved confounders U can be implied by the observational data X, e.g., the imaging data and non-imaging clinical information. Embodiments can recover the representations of U from the observed covariates X [19]-[25]. A challenge is that some observed covariates do not affect the outcome or treatment, which means they are not truly confounders. Specifically, these non-confounding covariates can be treated as noise in representation learning, which is especially common in high-dimensional data such as images. Conditioning on these non-confounding covariates can hurt the causal effect estimation [24]. To tackle this, embodiments specify the confounding effects in the high-dimensional multimodality feature space. By performing confounder distillation via auxiliary task of treatment assignment prediction, X effectively distills the covariates into the features relevant for the confounders U, yielding the representations Φ, which mainly contain the information of confounding variables.

Deep neural networks have achieved remarkable performance in outcome prediction. Nevertheless, it is still notoriously difficult to directly use deep learning methods for assisting treatment decision-making in clinical scenarios, due to the lack of explainability and robustness. To this end, embodiments of the subject invention perform outcome prediction from the perspective of treatment effect estimation, which can help find the optimal decision attributing to inferring causal effects of interventions. One of the challenges of causal effect estimation of this study is that the confounders cannot be directly observed. Although the representation learning can be of help to excavate the confounding factors from the observed data, there are existing some features implied on the observed covariates that can be considered as “noise” for the causal effect estimation as they are irrelevant for confounding. These noise sources are especially common in the high-dimensional data (e.g., the imaging data in the study below.) Therefore, the provided multi-modality confounder distillation can successfully improve the performance, attributing to forcing the model to focus on the confounding factors. Using this architecture means dropping some non-confounding features that only affect the outcome. This can result in certain embodiments exhibiting worse performance as a predictor for the outcome [24]. However, a task of the provided model is to estimate the discrepancy of outcomes with different treatments (e.g., Y (0)-Y (1)), instead of the outcome (Y (t)). Thus providing a beneficial trade-off for cases where clinicians should concentrate attention on the optimal treatment assignment instead of the final outcome. The provided experimental results also show that this architecture does not hurt the performance of outcome prediction in the provided task, and improves the performance of estimation to a great extent.

Another point of the example below is how to handle the systematic imbalance of the distributions of the covariates between different treatment groups. Although various methods have been proposed to solve this problem, there is no existing method focusing on studying this imbalance issue when integrating multi-modality data. Considering that balanced representation learning can be seen as one of the most effective algorithms to handle systematic imbalance in causal effect estimation, while multi-modality data naturally have sufficient information which can promote representation learning, certain embodiments combine the concepts of representation learning and multi-modality learning to achieve reliable causal effect estimation. Compared with the classic methods based on balanced presentation learning [13], [19], [22], the provided method utilizes the imaging and non-imaging interaction to assist the distribution balancing, that benefits from helping the model find the similar units with the knowledge extracted from different modalities.

It is contemplated within the scope of the subject invention that application of certain embodiments to one or more datasets that are larger and/or more balanced can produce further improvements in results. For example, using a dataset collected from randomized controlled trials, embodiments can better evaluate the provided model with true treatment effect. Cost and complexity to conduct randomized controlled trials in clinical scenarios are performance limiting factors, as is availability of related publicly available datasets. Certain data types (e.g., the noncontrast head CT data used in this example) can limit performance for treatment outcome prediction. In practice, the clinicians are strongly suggested to use CT angiography (CTA) or MR angiography (MRA) to comprehensively analyze the aneurysm for treatment management [2]. However, not all the cases have the data of CTA or MRA, while the noncontrast head CT are more frequently available as it remains the cornerstone of diagnosis of aSAH. Moreover, the clinically useful information are relatively hard for embodiments to extract from CTA and MRA, including the location of aneurysm, the aneurysm size, and whether the aneurysm is wide-necked. Thus, embodiments manually extract this information implied in CTA or MRA and use them as non-imaging data to perform the outcome prediction. In addition, as can be seen in Table III, the clinical non-imaging data can, in some cases, have more useful information than imaging data, as the performance of only using clinical data is better than that of only using imaging data. This can be attributed to the fact that the clinical non-imaging data actually contain some information extracted from CTA or MRA.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### Materials and Methods

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—Experiment

An embodiment of the subject invention is evaluated on a multi-modality clinical dataset of subarachnoid hemorrhage for treatment effect estimation. The experiments of comparison with related art methods and in-depth ablation study are performed successively.

An in-house dataset of subarachnoid hemorrhage collected from the Hospital Authority Data Collaboration Lab is employed. The dataset contains 656 cases with head CT scans and reported to have subarachnoid hemorrhage. Each case contains both CT imaging data and non-imaging clinical data. The non-imaging data have 16 clinical characteristics which have been shown to be potentially associated with the treatment outcome in clinical practice [10], [44], including gender, age, presence of hemorrhage of other subtypes, Glasgow Coma Scale, history of smoking and drinking, history of hypertension, history of diabetes, history of hyperlipidemia, pupil size, whether is wide-necked aneurysm, aneurysm size and location of aneurysm. Three types of treatments associated with aSAH are considered in this paper, including endovascular treatment, clipping, and conservative treatment. Patient survival after one year is considered to be the treatment outcome. Treatments are always performed separately, ensuring that the treatment does not have multiple forms or versions. The dataset contains 330 aSAH patients, among which 145 and 55 cases are treated by endovascular treatment and clipping respectively, and the others received conservative treatment (e.g., the patients who did not undergo any interventions according to doctor's recommendation or for other reasons). The dataset also contains 326 non-aneurysmal SAH patients. In clinical practice, only patients with aSAH are considered for treatment by endovascular treatment or clipping. This example uses the aSAH cases to train and test the outcome prediction task in the provided framework, while using both non-aneurysmal and aneurysmal cases to assist the treatment assignment task (e.g., to train the feature extraction networks Hing (⋅) by loss [symbol] ₚ in Equation. 5.) This example provides novel systems and methods comprising a publicly available dataset that contains multi-modality data and can be used to perform treatment outcome prediction.

Embodiments employ three evaluation metrics that are commonly used in causal effect estimation and treatment outcome prediction in the provided experiments, including the policy risk R_{POL} [19], the error in the average treatment effect ϵ_{ATE} [15], and the Accuracy Acc. The policy risk RPOL measures the average loss when using the treatment according to the treatment outcome estimator. Let f(x, t) be the outcome of treatment t estimated by the model, where x contains the covariates of the patient. Given the estimated treatment outcomes (e.g., the survival probability) f(x, 0), f(x, 1), and f(x, 2), π(x) denotes the treatment which has the best outcome. In other words, π(x) denotes the treatment recommended by the estimator. Then, the policy risk can be calculated by:

$${R}_{POL} ( π ) = 1 - ( 𝔼 [ Y ( 0 ) \vert π ( x ) = 0 ] \cdot 𝒫 ( π ( x ) = 0 ) + 𝔼 [ Y ( 1 ) \vert π ( x ) = 1 ] \cdot 𝒫 ( π ( x ) = 1 ) + 𝔼 [ Y ( 2 ) \vert π ( x ) = 2 ] \cdot 𝒫 ( π ( x ) = 2 ) ) , \qquad ( 14 )$$

and thus provides a lower-is-better evaluation metric.

Regarding the error in the average treatment effect CATE, it measures the distance between the true average treatment effect and the estimated average treatment effect. Let us take the ϵ_{ATE0,1} for example, which is the error in the average treatment effect of treatment T=0 and T=1. The true average treatment effect can be calculated by

$${ATE}_{0 , 1} = \frac{1}{\vert {S}_{0} \vert} {\sum }_{n ∈ {S}_{0}} {y}_{n} - \frac{1}{\vert {S}_{1} \vert} {\sum }_{n ∈ {S}_{1}} {y}_{n} ,$$

where S₀ and S₁ denote the set of receiving treatment T=0 and T=1 respectively, and [symbol] n denotes the factual outcome of the n-th sample. Then the error in the average treatment effect on the treated patient is

$${ϵ}_{ATE0 , 1} = \vert {ATE}_{0 , 1} - \frac{1}{\vert {S}_{0} \vert + \vert {S}_{1} \vert} {\sum }_{n ∈ {S}_{0} ⋃ {S}_{1}} ( f ( {x}_{n} , 0 ) - f ( {x}_{n} , 1 ) \vert .$$

Similarly, a lower score is better regarding the error in the average treatment effect ϵ_{ATE}. Moreover, embodiments use the factual outcomes to calculate the accuracy Acc in this study. For example, Acc₀ is calculated by only using the samples treated with treatment T=0.

Regarding the preprocessing of imaging data, the intensity values of raw images are truncated to the range of [−20, 100], and are further normalized to zero mean and unit variance. Each respective slice in the axial plane is resized to 224×224. As there exists dimensionality mismatch between imaging data and non-imaging data, embodiments use a 3D ResNet [45] to provide the feature extraction network H^{img}(⋅) for imaging data and certain embodiments remove the fully connected layer of ResNet-34 to maintain dimensional consistency. For the feature extraction of nonimaging clinical data, embodiments use three blocks of a fully connected layer followed by Batch Normalization layer and ReLU function to be the network H^{cli}. The provided model is implemented with PyTorch and run on a NVIDIA A100 SXM4 card. Embodiments trained the models using SGD with a momentum 0.9, a weight decay 5×10⁻³, and an initial learning rate 5×10⁻³. The training epoch is 1500 and the batch size is 128. The final results are the average of 10-fold cross validation. The training process lasted approximately 4 hours, while during the test phase, it took around 2 seconds per patient. Code is publicly available at https://github.com/med-air/TOP-aSAH.

For comparison with related art systems and methods, the provided method is first compared with the existing approaches for causal effect estimation as strong competitors, including BNN [13], which is a representative work on balancing the distributions of different treatment groups via discrepancy distance minimization, CFR-MMD [19], which proposes to use separate heads to estimate the causal effect and use Maximum Mean Discrepancy to balance the distribution, CFR-WASS [19], which uses the same separate heads with CFR-MMD but uses Wasserstein distance to balance the distributions, SITE [22], which focuses on hard samples to preserve local similarity and balances data distribution, Dragonnet [24], which uses propensity score to extract the confounding factors, and β-Intact-VAE [25], which proposes a new type of VAE to recover a prognostic score to help causal effect estimation. Notably, these methods are all proposed to estimate causal effect on single-modality data. To adapt these methods to the provided multi-modality data, the same multimodality feature extraction architectures are used as in the provided method (e.g., the networks Hᵢ(⋅) and Hⱼ(⋅).). Then the features extracted from these networks are concatenated and carefully follow the corresponding architectures described methods are followed to implement to each respective related art method.

The provided model according to an embodiment of the subject invention is compared with other multimodal methods (e.g., imaging data and tabular data). Specifically, the provided method is compared with DAFT [46], which is designed to suppress high-level concepts from 3D images while considering both image and tabular data, FILM [47], which utilizes FILM layer in the network to incorporate non-imaging data for visual reasoning, and Interactive-Model [48], which utilizes channel-wise multiplication of the intermediate features of imaging and non-imaging data to achieve the interaction between image and non-image data.

Table I presents the comparison results of different methods for aSAH treatment outcome estimation. The provided method according to an embodiment of the subject invention significantly improves the model performance over other methods regarding all the three evaluation metrics. This benefits from the provided multi-modality confounder distillation to emphasize features that affect both the treatment decision and the treatment outcome, and the provided imaging and non-imaging interaction representation learning to balance the distributions of different treatments. Compared with the classic methods based on balanced presentation learning, i.e., BNN [13] and CFRMMD/CFR-WASS [19], SITE not only balances the distributions of different treatment groups, but also preserves the local similarity, so that improves the estimation performance. Further, compared with SITE [22], the provided method not only uses a different strategy to achieve distribution balancing, but also uses the multi-modality information to select pairs and better utilize the complementary knowledge extracted from different modalities. For these reasons, the provided method improves the performances by 1.5% on R_{POL} and 2.0%, 4.4%, and 2.1% on ϵ_{ATE0,1}, ϵ_{ATE0,2}, and ϵ_{ATE1,2} respectively. On the other hand, Dragonnet proposes to use the propensity-score head to make the model focus on the confounding factors, which affects both treatment decisionmaking and outcome. Compared with Dragonnet [24], the provided method uses the treatment assignment to select the pairs to promote the representation learning, improving the the performances by 2.0% on R_{POL} and 3.7%, 2.0%, and 4.5% on ϵ_{ATE0,1}, ϵ_{ATE0,2}, and ϵ_{ATE1,2} respectively. Moreover, Dragonnet can have better performance when there are numerous covariates which influence only the outcome [24]. This character can not apply equally well on the provided in-house dataset. Hence, the provided method can significantly improve the performance over Dragonnet. Moreover, compared with previous existing works, β-Intact-VAE is proved to be effective in estimating causal effect under limited overlap, which means not all the treatments t∈{0, 1, 2} have the possibility to be assigned to some particular patients. However, β-Intact-VAE seeks to use VAE model to encode the observed covariates X to a low-dimensional representations (i.e., 1-dimensional representations). Without joining learned multi-modality feature representations from intermediate layers, this strategy of simply combing two low-dimensional representations encoded from different modalities can lead to loss of complementary information. Thus, the provided model achieves better performance to varying degrees compared with β-Intact-VAE. In addition, compared to the specialized multi-modal classification methods, the provided method achieved the best results. This is due to the fact that the provided method not only considers the interaction between multiple modalities but also improves the model's predictive performance for the outcome of all treatment assignments from the perspective of causal effect estimation.

Analysis of the provided method according to an embodiment of the subject invention is provided in four primary areas, as follows.

1) Benefits of Multi-modality Data: first, the advantage of using multi-modality data for treatment outcome predictions in aSAH is demonstrated. Specifically, two additional models with the provided method are implemented by using only the imaging data (Image) or only the non-imaging clinical data (Clinical). Note that these two models are also equipped with the confounder distillation and balanced distribution learning strategies, but the features Φ extracted from the same modality are used to select pairs. As can be seen in Table III, the models using only imaging data or clinical non-imaging data exhibit reduced performance. Compared to the “Image” and “Clinical” model, using the multi-modality data in the provided method can help improve the policy risk from 10.6% or 9.8% to 5.8%. This can be attributed to the fact that the multi-modality data have rich complementary information, and with the distilled confounders, the imaging and non-imaging interaction achieves more balanced representation learning, demonstrating the advantageous benefits of combining multi-modality data according to this embodiment of the subject invention.

2) Contribution of Each Component: the contribution of the provided multi-modality confounders distillation (MMCD) and the balanced representation learning (BRL) are analyzed via imaging and non-imaging interaction, by adding only either one of them, respectively, onto the baseline. As can be seen in Table IV, adding only MMCD or INII improves the policy risk from the baseline results 10.1% to 8.4%, and 7.4% respectively, showing that each component plays their role in the causal effect estimation. Compared with the MMCD model, the provided provided method can further improve the performance by 2.6% on R_{POL}, 4.1% on ϵ̅_{ATE}, and 2.7% on Acc. These improvements attribute to the imaging and non-imaging interaction to assist in balancing the distributions of samples with different treatments. Compared with the INII model, the provided method can improve the performance by 1.6% on R_{POL}, 3.0% on ϵ̅_{ATE}, and 1.3% on Acc, indicating that the distilled confounding factors can help guide the imaging and nonimaging interaction for balanced representation learning.

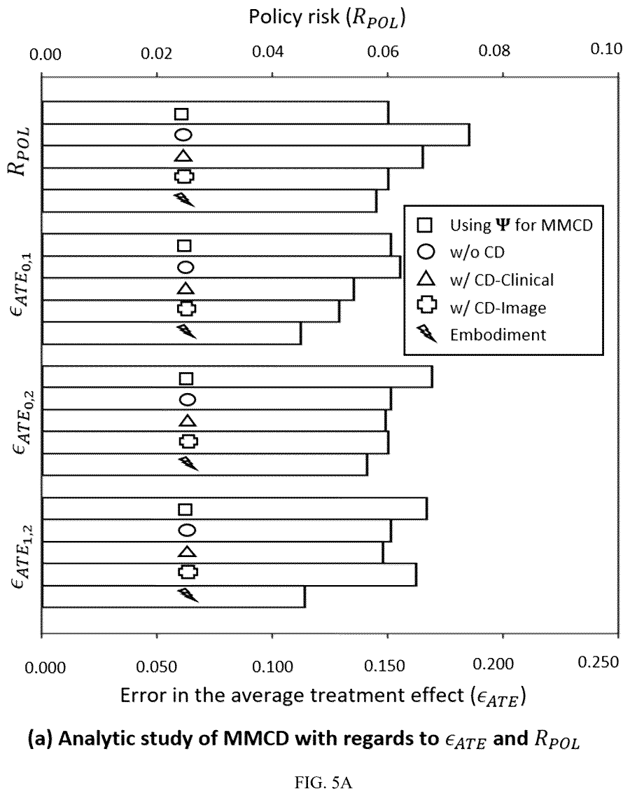

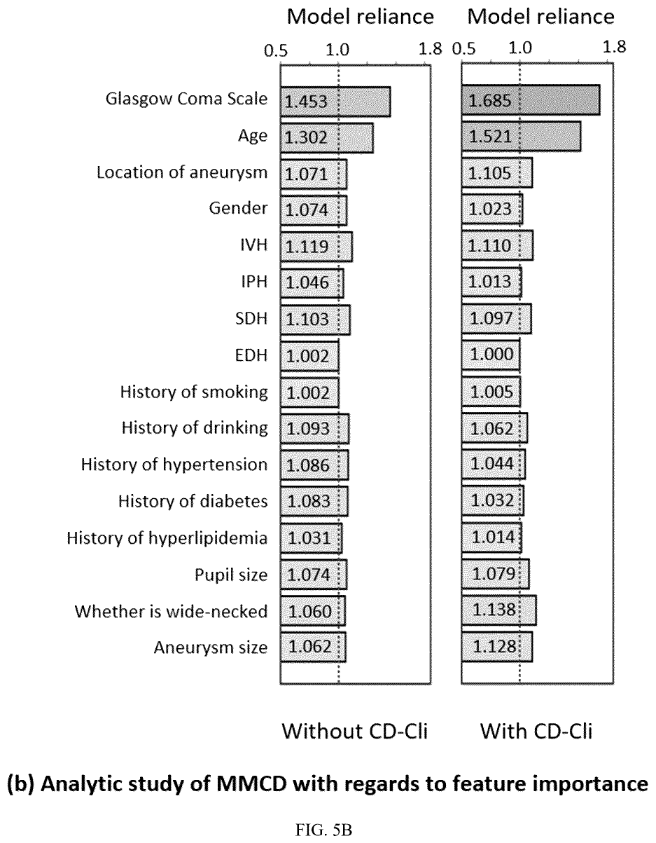

3) Effect of Confounders Distillation: more in-depth analysis of the effect of the provided multi-modality confounder distillation is further conducted. As can be seen in FIG. 5A, either adding the confounder distillation via treatment assignment prediction on the imaging data branch (e.g., w/CD-Image Only, as shown in FIG. 2B or on the non-imaging clinical data branch (w/CD-Clinical Only, as shown in FIG. 2C) benefits model performance, but “w/CD-Image Only” presents more obvious improvements. This indicates that the treatment assignment prediction is of great value in distilling the confounders from each modality, especially for the imaging data which are high-dimensional thus are in more need of confounders distillation. Additionally, an ablation study is conducted to analyze which part of the provided model is more suitable for the insertion of the MMCD module (e.g., Φ or Ψ). The results shown in FIG. 5A demonstrate that using the Φ feature (i.e., as shown in FIG. 2A) will yield better performance (i.e., as shown in the series labeled Embodiment in FIG. 5A). This can be attributed to the different feature requirements of MMCD and the final treatment effect estimation tasks. Specifically, the former performs better with greater difference in the features of data from different treatments, while the latter performs better with greater overlap. The lowest error in each measurement group comes from the Embodiment as shown in FIG. 2A (i.e., the bottom (green, lightning bolt) bar in each group.)

Furthermore, the importance of each nonimaging clinical covariate on the outcome prediction with the permutation importance technique is estimated [49]. Specifically, model reliance is used as the metric to measure the covariate importance. The higher model reliance of a covariate means the model depends more on that covariate. FIG. 5B shows that by adding the confounder distillation on the clinical data, the model reliance in age, Glasgow Coma Scale, and location of aneurysm greatly increases, which aligns with the clinical findings on the important covariates affecting both treatment and outcome [2]. These results validate that using treatment assignment prediction as the auxiliary task can successfully help distill the useful information.

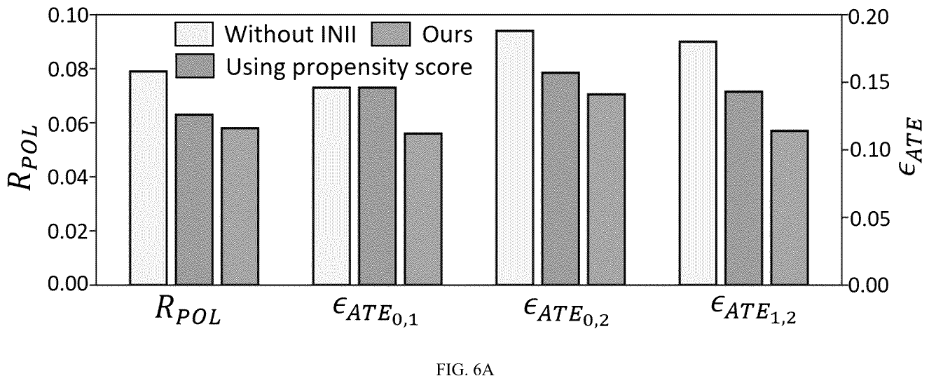

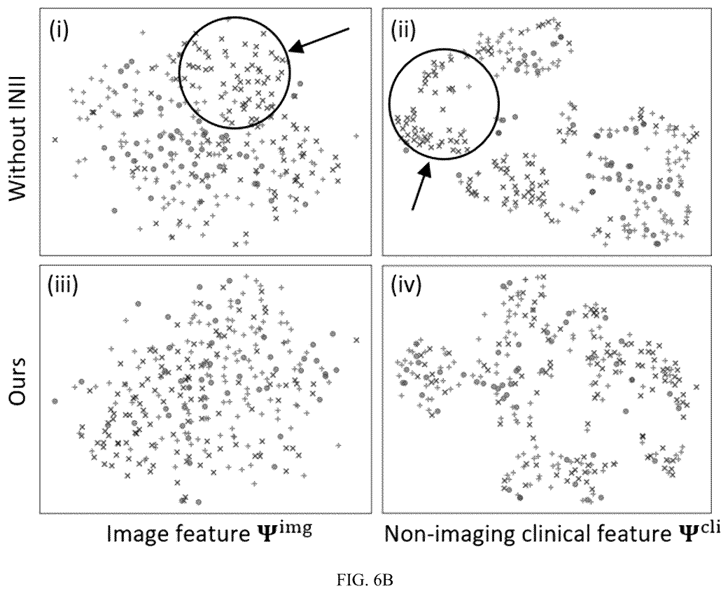

4) Importance of Imaging and Non-imaging Interaction: To analyze the necessity of imaging and non-imaging interaction (INII) to guide the balanced distribution learning, the other implementations are hold the same as the provided method but directly use the features Φ extracted from the same modality to select pairs for distribution balancing, as shown in FIG. 2E. As can be seen in FIG. 6A, the provided method with INII achieves better performance as expected, benefiting from the fact that the imaging and non-imaging interaction representation learning can utilize rich complementary information extracted from different modalities to help distribution balancing. The t-SNE visualization of the learned representations is shown in FIG. 6B. For features learned without INII in FIGS. 6B(i) and 6B(ii), there are obvious non-overlap regions (see the regions of the black circles), resulting in unreliable counterfactual estimation for treatment outcome prediction. By incorporating the INII design, the provided method learns more balanced feature distributions with increased overlap between different treatment groups (see FIGS. 6B(i) and 6B(ii)), benefiting the causal effect estimation.

Embodiment 1. A machine learning system for providing treatment decision support from imaging data and clinical data with causal effect estimation and a known number of treatment options (i) where i is a positive integer, the system comprising:
an image feature extraction network (H^{img}(⋅)) configured to process an image dataset to produce an image feature (img) extract;
an image prediction network (G^{img}(⋅)) configured to produce an image-predicted treatment assignment (\hat{t}_{img}) from the image feature extract;
a first image feature development network (C^{img}(⋅)) configured to produce a first image feature representation (img) from the image feature extract; and
a plurality of second image feature development networks (Di^{img}(⋅)) each respectively configured to produce a respective second image outcome representation (Ψ̅i^{img}) from the first image feature representation.

Embodiment 2. The system according to Embodiment 1, further comprising:
a clinical feature extraction network (H^{cli} (⋅)) configured to process a clinical dataset to produce a clinical feature (Φ^{cli}) extract;
a clinical prediction network (G^{cli}(⋅)) configured to produce a clinical-predicted treatment assignment (\hat{t}^{cli}) from the clinical feature extract;
a first clinical feature development network (C^{cli}(⋅)) configured to produce a first clinical feature representation (Ψ^{cli}) from the clinical feature extract; and
a plurality of second clinical feature development networks (Di^{cli}(⋅)) each respectively configured to produce a respective second clinical outcome representation (Ψ̅ᵢ^{cli}) from the first clinical feature representation.

Embodiment 3. The system according to Embodiment 2, further comprising:
an imaging and non-imaging interaction (INII) operator configured to enable a model to learn balanced image feature representation (Ψ^{img}) and the first clinical feature representation (Ψ^{cli}).

Embodiment 4. The system according to Embodiment 3, further comprising:
a concatenator configured to produce a respective survival probability for each treatment option (i) from the corresponding second image outcome representation (Ψ̅i^{img}) and the corresponding respective second clinical outcome representation (Ψ̅ᵢ^{cli}).

Embodiment 5. The system according to Embodiment 4, further comprising:
a treatment recommender configured to produce a treatment recommendation from the outcomes of different treatment assignments (ŷ₀, ŷ₁ and ŷ₂).

Embodiment 6. The system according to embodiment 1, wherein the image feature extraction network (H^{img}(⋅)) is configured to highlight information of confounders that are defined as parts of inputs affecting both treatment assignment and treatment outcome.

Embodiment 7. The system according to embodiment 6, wherein the confounders of each modality are distilled from inputs, while factors irrelevant for treatment are attenuated.

Embodiment 8. A machine learning method for providing treatment decision support from imaging data and clinical data with causal effect estimation and a known number of treatment options (i) where i is a positive integer, the method comprising:
configuring an image feature extraction network (H^{img}(⋅)) to process an image dataset to produce an image feature (Ψ^{img}) extract;
configuring an image prediction network (G^{img}(⋅)) to produce an image-predicted treatment assignment (\hat{t}^{img}) from the image feature extract;
configuring a first image feature development network (C^{img}(⋅)) to produce a first image feature representation (Ψ^{img}) from the image feature extract; and
configuring each of a plurality of second image feature development networks (Di^{img}(⋅)) to produce a respective second image outcome representation (Ψ̅ᵢ^{img}) from the first image feature representation.

Embodiment 9. The method according to Embodiment 8, further comprising:
configuring a clinical feature extraction network (H^{cli} (⋅)) to process a clinical dataset to produce a clinical feature (Φ^{cli}) extract;
configuring a clinical prediction network (G^{cli}(⋅)) to produce a clinical-predicted treatment assignment (\hat{t}^{cli}) from the clinical feature extract;
configuring a first clinical feature development network (C^{cli}(⋅)) to produce a first clinical feature representation (Ψ^{cli}) from the clinical feature extract; and
configuring each of a plurality of second clinical feature development networks (Di^{cli}(⋅)) to produce a respective second clinical outcome representation (Ψ̅ᵢ^{cli}) from the first clinical feature representation.

Embodiment 10. The method according to Embodiment 9, further comprising:
configuring an imaging and non-imaging interaction (INII) operator configured to enable a model to learn balanced image feature representation (Ψ^{img}) and the first clinical feature representation (Ψ^{cli}).

Embodiment 11. The method according to Embodiment 10, further comprising:
configuring a concatenator to produce a respective survival probability for each treatment option (i) from the corresponding second image outcome representation (Ψ̅ᵢ^{img}) and the corresponding respective second clinical outcome representation (Ψ̅ᵢ^{cli}).

Embodiment 12. The method according to Embodiment 11, further comprising:
configuring a treatment recommender to produce a treatment recommendation from the outcomes of different treatment assignments (ŷ₀, ŷ₁ and ŷ₂).

Embodiment 13. The system according to claim 8, wherein the image feature extraction network (H^{img}(⋅)) is configured to highlight information of confounders that are defined as parts of inputs affecting both treatment assignment and treatment outcome.

Embodiment 14. The system according to claim 13, wherein the confounders of each modality are distilled from inputs, while factors irrelevant for treatment are attenuated.

Embodiment 15. A computer program product, comprising:
a non-transitory computer-executable storage device having computer readable program instructions embodied thereon that when executed by a computer cause the computer to perform a machine learning method for providing treatment decision support from imaging data and clinical data with causal effect estimation and a known number of treatment options (i) where i is a positive integer, the computer-executable program instruction comprising:

configuring an image feature extraction network (H^{img}(⋅)) to process an image dataset to produce an image feature (Φ^{img}) extract;
configuring an image prediction network (G^{img}(⋅)) to produce an image-predicted treatment assignment (\hat{t}^{img}) from the image feature extract;
configuring a first image feature development network (C^{img}(⋅)) to produce a first image feature representation (Ψ^{img}) from the image feature extract; and
configuring each of a plurality of second image feature development networks (Di^{img}(⋅)) to produce a respective second image outcome representation (Ψ̅ᵢ^{img}) from the first image feature representation.

Embodiment 16. The computer program product according to Embodiment 15, further comprising:
configuring a clinical feature extraction network (H^{cli} (⋅)) to process a clinical dataset to produce a clinical feature (Φ^{cli}) extract;
configuring a clinical prediction network (G^{cli}(⋅)) to produce a clinical-predicted treatment assignment (\hat{t}^{cli}) from the clinical feature extract;
configuring a first clinical feature development network (C^{cli}(⋅)) to produce a first clinical feature representation (Ψ^{cli}) from the clinical feature extract; and
configuring each of a plurality of second clinical feature development networks (Dᵢ^{cli}(⋅)) to produce a respective second clinical outcome representation (Ψ̅ᵢ^{cli}) from the first clinical feature representation.

Embodiment 17. The computer program product according to Embodiment 16, further comprising:
configuring an imaging and non-imaging interaction (INII) operator configured to enable a model to learn balanced image feature representation (Ψ^{img}) and the first clinical feature representation (Ψ^{cli}).

Embodiment 18. The computer program product according to Embodiment 17, further comprising:
configuring a concatenator to produce a respective survival probability for each treatment option (i) from the corresponding second image outcome representation (Ψ̅ᵢ^{img}) and the corresponding respective second clinical outcome representation (Ψ̅ᵢ^{cli}).

Embodiment 19. The computer program product according to Embodiment 18, further comprising:
configuring a treatment recommender to produce a treatment recommendation from the outcomes of different treatment assignments (ŷ₀, ŷ₁ and ŷ₂).

Embodiment 20. The system according to embodiment 15, wherein the image feature extraction network (H^{img}(⋅)) is configured to highlight information of confounders that are defined as parts of inputs affecting both treatment assignment and treatment outcome.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

[1] D. J. Nieuwkamp, L. E. Setz, A. Algra, F. H. Linn, N. K. de Rooij, and G. J. Rinkel, “Changes in case fatality of aneurysmal subarachnoid haemorrhage over time, according to age, sex, and region: a metaanalysis,” The Lancet Neurology, vol. 8, no. 7, pp. 635-642, 2009.
[2] E. S. Connolly Jr, A. A. Rabinstein, J. R. Carhuapoma, C. P. Derdeyn, J. Dion, R. T. Higashida, B. L. Hoh, C. J. Kirkness, A. M. Naidech, C. S. Ogilvy, et al., “Guidelines for the management of aneurysmal subarachnoid hemorrhage: a guideline for healthcare professionals from the american heart association/american stroke association,” Stroke, vol. 43, no. 6, pp. 1711-1737, 2012.
[3] S. H. Chang, H. S. Shin, S. H. Lee, H. C. Koh, and J. S. Koh, “Rebleeding of ruptured intracranial aneurysms in the immediate postoperative period after coil embolization,” Journal of Cerebrovascular and Endovascular Neurosurgery, vol. 17, no. 3, pp. 209-216, 2015.
[4] A. Lindgren, M. D. Vergouwen, I. van der Schaaf, A. Algra, M. Wermer, M. J. Clarke, and G. J. Rinkel, “Endovascular coiling versus neurosurgical clipping for people with aneurysmal subarachnoid haemorrhage,” Cochrane Database of Systematic Reviews, no. 8, 2018.
[5] C. E. van Donkelaar, N. A. Bakker, J. Birks, N. J. Veeger, J. D. Metzemaekers, A. J. Molyneux, R. J. Groen, and J. M. C. van Dijk, “Prediction of outcome after aneurysmal subarachnoid hemorrhage: development and validation of the safire grading scale,” Stroke, vol. 50, no. 4, pp. 837-844, 2019.
[6] M. Katsuki, Y. Kakizawa, A. Nishikawa, Y. Yamamoto, and T. Uchiyama, “Postsurgical functional outcome prediction model using deep learning framework (prediction one, sony network communications inc.) for hypertensive intracerebral hemorrhage,” Surgical Neurology International, vol. 12, 2021.
[7] C. Rubbert, K. R. Patil, K. Beseoglu, C. Mathys, R. May, M. G. Kaschner, B. Sigl, N. A. Teichert, J. Boos, B. Turowski, et al., “Prediction of outcome after aneurysmal subarachnoid haemorrhage using data from patient admission,” European radiology, vol. 28, no. 12, pp. 4949-4958, 2018.
[8] M. Katsuki, S. Kawamura, and A. Koh, “Easily created prediction model using automated artificial intelligence framework (prediction one, sony network communications inc., tokyo, japan) for subarachnoid hemorrhage outcomes treated by coiling and delayed cerebral ischemia,” Cureus, vol. 13, no. 6, 2021.
[9] P. de Toledo, P. M. Rios, A. Ledezma, A. Sanchis, J. F. Alen, and A. Lagares, “Predicting the outcome of patients with subarachnoid hemorrhage using machine learning techniques,” IEEE Transactions on Information Technology in Biomedicine, vol. 13, no. 5, pp. 794-801, 2009.
[10] J. Shen, J. Yu, S. Huang, R. Mungur, K. Huang, X. Pan, G. Yu, Z. Xie, L. Zhou, Z. Liu, et al., “Scoring model to predict functional outcome in poor-grade aneurysmal subarachnoid hemorrhage,” Frontiers in Neurology, vol. 12, p. 601996, 2021.
[11] G. de Jong, R. Aquarius, B. Sanaan, R. H. Bartels, J. A. Grotenhuis, D. J. Henssen, and H. D. Boogaarts, “Prediction models in aneurysmal subarachnoid hemorrhage: forecasting clinical outcome with artificial intelligence,” Neurosurgery, vol. 88, no. 5, pp. E427-E434, 2021.
[12] G. W. Imbens and D. B. Rubin, Causal inference in statistics, social, and biomedical sciences. Cambridge University Press, 2015.
[13] F. Johansson, U. Shalit, and D. Sontag, “Learning representations for counterfactual inference,” in ICML. PMLR, 2016, pp. 3020-3029.
[14] M. J. Lopez and R. Gutman, “Estimation of causal effects with multiple treatments: a review and new ideas,” Statistical Science, pp. 432-454, 2017.
[15] L. Hu, C. Gu, M. Lopez, J. Ji, and J. Wisnivesky, “Estimation of causal effects of multiple treatments in observational studies with a binary outcome,” Statistical methods in medical research, vol. 29, no. 11, pp. 3218-3234, 2020.
[16] J. Ma, R. Guo, A. Zhang, and J. Li, “Multi-cause effect estimation with disentangled confounder representation.” in IJCAI, 2021, pp. 2790-2796.
[17] J. Peters, D. Janzing, and B. Schölkopf, Elements of causal inference: foundations and learning algorithms. The MIT Press, 2017.
[18] H. Li, R. Pan, H. Wang, X. Rong, Z. Yin, D. P. Milgrom, X. Shi, Y. Tang, and Y. Peng, “Clipping versus coiling for ruptured intracranial aneurysms: a systematic review and meta-analysis,” Stroke, vol. 44, no. 1, pp. 29-37, 2013.
[19] U. Shalit, F. D. Johansson, and D. Sontag, “Estimating individual treatment effect: generalization bounds and algorithms,” in International conference on machine learning. PMLR, 2017, pp. 3076-3085.
[20] C. Louizos, U. Shalit, J. M. Mooij, D. Sontag, R. Zemel, and M. Welling, “Causal effect inference with deep latent-variable models,” Advances in neural information processing systems, vol. 30, 2017.
[21] P. Schwab, L. Linhardt, and W. Karlen, “Perfect match: A simple method for learning representations for counterfactual inference with neural networks,” arXiv preprint arXiv: 1810.00656, 2018.
[22] L. Yao, S. Li, Y. Li, M. Huai, J. Gao, and A. Zhang, “Representation learning for treatment effect estimation from observational data,” Advances in Neural Information Processing Systems, vol. 31, 2018.
[23] J. Yoon, J. Jordon, and M. Van Der Schaar, “Ganite: Estimation of individualized treatment effects using generative adversarial nets,” in International Conference on Learning Representations, 2018.
[24] C. Shi, D. Blei, and V. Veitch, “Adapting neural networks for the estimation of treatment effects,” Advances in Neural Information Processing Systems, vol. 32, 2019.
[25] P. Wu and K. Fukumizu, “\beta-intact-vae: Identifying and estimating causal effects under limited overlap,” ICLR, 2022.
[26] S. Yang and P. Ding, “Asymptotic inference of causal effects with observational studies trimmed by the estimated propensity scores,” Biometrika, vol. 105, no. 2, pp. 487-493, 2018.
[27] T. B. Armstrong and M. Koles'ar, “Finite-sample optimal estimation and inference on average treatment effects under unconfoundedness,” Econometrica, vol. 89, no. 3, pp. 1141-1177, 2021.
[28] E. A. Stuart, “Matching methods for causal inference: A review and a look forward,” Statistical science: a review journal of the Institute of Mathematical Statistics, vol. 25, no. 1, p. 1, 2010.
[29] P. C. Austin, “An introduction to propensity score methods for reducing the effects of confounding in observational studies,” Multivariate behavioral research, vol. 46, no. 3, pp. 399-424, 2011.
[30] P. R. Rosenbaum, “Modern algorithms for matching in observational studies,” Annual Review of Statistics and Its Application, vol. 7, pp. 143-176, 2020.
[31] J. T. Senders, P. C. Staples, A. V. Karhade, M. M. Zaki, W. B. Gormley, M. L. Broekman, T. R. Smith, and O. Arnaout, “Machine learning and neurosurgical outcome prediction: a systematic review,” World neurosurgery, vol. 109, pp. 476-486, 2018.
[32] G. Brugnara, U. Neuberger, M. A. Mahmutoglu, M. Foltyn, C. Herweh, S. Nagel, S. Schönenberger, S. Heiland, C. Ulfert, P. A. Ringleb, et al., “Multimodal predictive modeling of endovascular treatment outcome for acute ischemic stroke using machine-learning,” Stroke, vol. 51, no. 12, pp. 3541-3551, 2020.
[33] A. Nielsen, M. B. Hansen, A. Tietze, and K. Mouridsen, “Prediction of tissue outcome and assessment of treatment effect in acute ischemic stroke using deep learning,” Stroke, vol. 49, no. 6, pp. 1394-1401, 2018.
[34] H.-L. Wang, W.-Y. Hsu, M.-H. Lee, H.-H. Weng, S.-W. Chang, J.-T. Yang, and Y.-H. Tsai, “Automatic machine-learning-based outcome prediction in patients with primary intracerebral hemorrhage,” Frontiers in neurology, vol. 10, p. 910, 2019.
[35] S.-C. Huang, A. Pareek, S. Seyyedi, I. Banerjee, and M. P. Lungren, “Fusion of medical imaging and electronic health records using deep learning: a systematic review and implementation guidelines,” NPJ digital medicine, vol. 3, no. 1, pp. 1-9, 2020.
[36] M. J. Funk, D. Westreich, C. Wiesen, T. Sturmer, M. A. Brookhart, and M. Davidian, “Doubly robust estimation of causal effects,” American journal of epidemiology, vol. 173, no. 7, pp. 761-767, 2011.
[37] P. R. Rosenbaum and D. B. Rubin, “The central role of the propensity score in observational studies for causal effects,” Biometrika, vol. 70, no. 1, pp. 41-55, 1983.
[38] R. K. Crump, V. J. Hotz, G. W. Imbens, and O. A. Mitnik, “Nonparametric tests for treatment effect heterogeneity,” The Review of Economics and Statistics, vol. 90, no. 3, pp. 389-405, 2008.
[39] Y. Chang and J. Dy, “Informative subspace learning for counterfactual inference,” in AAAI, vol. 31, no. 1, 2017.
[40] H. A. Chipman, E. I. George, and R. E. McCulloch, “Bart: Bayesian additive regression trees,” The Annals of Applied Statistics, vol. 4, no. 1, pp. 266-298, 2010.
[41] S. Athey and G. Imbens, “Recursive partitioning for heterogeneous causal effects,” Proceedings of the National Academy of Sciences, vol. 113, no. 27, pp. 7353-7360, 2016.
[42] S. Wager and S. Athey, “Estimation and inference of heterogeneous treatment effects using random forests,” Journal of the American Statistical Association, vol. 113, no. 523, pp. 1228-1242, 2018.
[43] T. Chen, S. Kornblith, M. Norouzi, and G. Hinton, “A simple framework for contrastive learning of visual representations,” in International conference on machine learning. PMLR, 2020, pp. 1597-1607.
[44] S. Chilamkurthy, R. Ghosh, S. Tanamala, M. Biviji, N. G. Campeau, V. K. Venugopal, V. Mahajan, P. Rao, and P. Warier, “Deep learning algorithms for detection of critical findings in head ct scans: a retrospective study,” The Lancet, vol. 392, no. 10162, pp. 2388-2396, 2018.
[45] K. He, X. Zhang, S. Ren, and J. Sun, “Deep residual learning for image recognition,” in CVPR, 2016, pp. 770-778.
[46] S. P″olsterl, T. N. Wolf, and C. Wachinger, “Combining 3d image and tabular data via the dynamic affine feature map transform,” in International Conference on Medical Image Computing and Computer-Assisted Intervention. Springer, 2021, pp. 688-698.
[47] E. Perez, F. Strub, H. De Vries, V. Dumoulin, and A. Courville, “Film: Visual reasoning with a general conditioning layer,” in Proceedings of the AAAI conference on artificial intelligence, vol. 32, no. 1, 2018.
[48] H. Duanmu, P. B. Huang, S. Brahmavar, S. Lin, T. Ren, J. Kong, F. Wang, and T. Q. Duong, “Prediction of pathological complete response to neoadjuvant chemotherapy in breast cancer using deep learning with integrative imaging, molecular and demographic data,” in International conference on medical image computing and computerassisted intervention. Springer, 2020, pp. 242-252.
[49] A. Fisher, C. Rudin, and F. Dominici, “All models are wrong, but many are useful: Learning a variable's importance by studying an entire class of prediction models simultaneously.” J. Mach. Learn. Res., vol. 20, no. 177, pp. 1-81, 2019.
[50] L. Van der Maaten and G. Hinton, “Visualizing data using t-sne.” Journal of machine learning research, vol. 9, no. 11, 2008.

We claim:

1. A machine learning system for providing treatment decision support from imaging data and clinical data with causal effect estimation and a known number of treatment options (i) where i is a positive integer, the system comprising:
an image feature extraction network (H^{img}(⋅)) configured to process an image dataset to produce an image feature (Φ^{img}) extract;
an image prediction network (G^{img}(⋅)) configured to produce an image-predicted treatment assignment (\hat{t}^{img}) from the image feature extract;
a first image feature development network (C^{img}(⋅)) configured to produce a first image feature representation (Ψ^{img}) from the image feature extract; and
a plurality of second image feature development networks (Di^{img}(⋅)) each respectively configured to produce a respective second image outcome representation (Ψ̅i^{img}) from the first image feature representation.

2. The system according to claim 1, further comprising:
a clinical feature extraction network (H^{cli} (⋅)) configured to process a clinical dataset to produce a clinical feature (Φ^{cli}) extract;
a clinical prediction network (G^{cli}(⋅)) configured to produce a clinical-predicted treatment assignment (\hat{t}^{cli}) from the clinical feature extract;
a first clinical feature development network (C^{cli}(⋅)) configured to produce a first clinical feature representation (Ψ^{cli}) from the clinical feature extract; and
a plurality of second clinical feature development networks (Di^{cli} (⋅)) each respectively configured to produce a respective second clinical outcome representation (Ψ̅ᵢ^{cli}) from the first clinical feature representation.

3. The system according to claim 2, further comprising:
an imaging and non-imaging interaction (INII) operator configured to enable a model to learn balanced image feature representation (Ψ^{img}) and the first clinical feature representation (Ψ^{cli}).

4. The system according to claim 3, further comprising:
a concatenator configured to produce a respective survival probability for each treatment option (i) from the corresponding second image outcome representation (Ψ̅i^{img}) and the corresponding respective second clinical outcome representation (Ψ̅ᵢ^{cli}).

5. The system according to claim 4, further comprising:
a treatment recommender configured to produce a treatment recommendation from the outcomes of different treatment assignments (ŷ₀, ŷ₁ and ŷ₂).

6. The system according to claim 1, wherein the image feature extraction network (H^{img}(⋅)) is configured to highlight information of confounders that are defined as parts of inputs affecting both treatment assignment and treatment outcome.

7. The system according to claim 6, wherein the confounders of each modality are distilled from inputs, while factors irrelevant for treatment are attenuated.

8. A machine learning method for providing treatment decision support from imaging data and clinical data with causal effect estimation and a known number of treatment options (i) where i is a positive integer, the method comprising:
configuring an image feature extraction network (H^{img}(⋅)) to process an image dataset to produce an image feature (Ψ^{img}) extract;
configuring an image prediction network (G^{img}(⋅)) to produce an image-predicted treatment assignment (\hat{t}^{img}) from the image feature extract;
configuring a first image feature development network (C^{img}(⋅)) to produce a first image feature representation (Ψ^{img}) from the image feature extract; and
configuring each of a plurality of second image feature development networks (Di^{img}(⋅)) to produce a respective second image outcome representation (Ψi^{img}) from the first image feature representation.

9. The method according to claim 8, further comprising:
configuring a clinical feature extraction network (H^{cli} (⋅)) to process a clinical dataset to produce a clinical feature (Φ^{cli}) extract;
configuring a clinical prediction network (G^{cli}(⋅)) to produce a clinical-predicted treatment assignment (\hat{t}^{cli}) from the clinical feature extract;
configuring a first clinical feature development network (C^{cli}(⋅)) to produce a first clinical feature representation (Ψ^{cli}) from the clinical feature extract; and
configuring each of a plurality of second clinical feature development networks (Di^{cli}(⋅)) to produce a respective second clinical outcome representation (Ψ̅ᵢ^{cli}) from the first clinical feature representation.

10. The method according to claim 9, further comprising:
configuring an imaging and non-imaging interaction (INII) operator configured to enable a model to learn balanced image feature representation (Ψ^{img}) and the first clinical feature representation (Ψ^{cli}).

11. The method according to claim 10, further comprising:
configuring a concatenator to produce a respective survival probability for each treatment option (i) from the corresponding second image outcome representation (Ψ̅i^{img}) and the corresponding respective second clinical outcome representation (Ψ̅ᵢ^{cli}).

12. The method according to claim 11, further comprising:
configuring a treatment recommender to produce a treatment recommendation from the outcomes of different treatment assignments (ŷ₀, ŷ₁ and ŷ₂).

13. The method according to claim 8, wherein the image feature extraction network (H^{img}(⋅)) is configured to highlight information of confounders that are defined as parts of inputs affecting both treatment assignment and treatment outcome.

14. The method according to claim 13, wherein the confounders of each modality are distilled from inputs, while factors irrelevant for treatment are attenuated.

15. A computer program product, comprising:
a non-transitory computer-executable storage device having computer readable program instructions embodied thereon that when executed by a computer cause the computer to perform a machine learning method for providing treatment decision support from imaging data and clinical data with causal effect estimation and a known number of treatment options (i) where i is a positive integer, the computer-executable program instruction comprising:
configuring an image feature extraction network (H^{img}(⋅)) to process an image dataset to produce an image feature (Φ^{img}) extract;
configuring an image prediction network (G^{img}(⋅)) to produce an image-predicted treatment assignment (\hat{t}^{img}) from the image feature extract;
configuring a first image feature development network (C^{img}(⋅)) to produce a first image feature representation (Ψ^{img}) from the image feature extract; and
configuring each of a plurality of second image feature development networks (Di^{img}(⋅)) to produce a respective second image outcome representation (Ψ̅i^{img}) from the first image feature representation.

16. The computer program product according to claim 15, further comprising:
configuring a clinical feature extraction network (H^{cli} (⋅)) to process a clinical dataset to produce a clinical feature (Φ^{cli}) extract;
configuring a clinical prediction network (G^{cli}(⋅)) to produce a clinical-predicted treatment assignment (\hat{t}^{cli}) from the clinical feature extract;
configuring a first clinical feature development network (C^{cli}(⋅)) to produce a first clinical feature representation (Ψ^{cli}) from the clinical feature extract; and
configuring each of a plurality of second clinical feature development networks (Di^{cli}(⋅)) to produce a respective second clinical outcome representation (Ψ̅ᵢ^{cli}) from the first clinical feature representation.

17. The computer program product according to claim 16, further comprising:
configuring an imaging and non-imaging interaction (INII) operator configured to enable a model to learn balanced image feature representation (Ψ^{img}) and the first clinical feature representation (Ψ^{cli}).

18. The computer program product according to claim 17, further comprising:
configuring a concatenator to produce a respective survival probability for each treatment option (i) from the corresponding second image outcome representation (Ψ̅i^{img}) and the corresponding respective second clinical outcome representation (Ψ̅ᵢ^{cli}).

19. The computer program product according to claim 18, further comprising:
configuring a treatment recommender to produce a treatment recommendation from the outcomes of different treatment assignments (ŷ₀, ŷ₁ and ŷ₂).

20. The computer program product according to claim 15, wherein the image feature extraction network (H^{img}(⋅)) is configured to highlight information of confounders that are defined as parts of inputs affecting both treatment assignment and treatment outcome.
